SEQUENCE LISTING (2 sequences)

SEQ ID NO: 1 - protein, 277 aa, Human CD40
MVRLPLQCVLWGCLLTAVHPEPPTACREKQYLINSQCCSLCQPGQKLVSDCTEFTETECLPCGESEFLDTWDRETHCHQHKYCDPNLGLRVQQKGTSETDTICTCEEGWHCTSEACESCVLHRSCSPGFGVKQIATGVSDTICEPCPVGFFSNVSSAGEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 12 aa, HUMAN
TEFTETECLPCG

(19) United States
(12) Patent Application Publication
(10) Pub. No.: US 2002/0031512 A1
(43) Pub. Date: Mar. 14, 2002

(54) CD40 antagonists for use in treating psoriasis and other inflammatory skin conditions

(76) Inventors: M. C. Pasch, Almere (NL); J.D. Bos, Heemstede (NL); David Thomas, Houston, TX (US)

Correspondence Address: TANOX, INC., 10301 STELLA LINK, HOUSTON, TX, 77025, US

(21) Appl. No.: 09/839,339

(22) Filed: Apr. 19, 2001

Related U.S. Application Data
(60) Provisional application No. 60/198,174, filed on Apr. 19, 2000.

(51) Int. Cl. A61K 39/395

(52) U.S. Cl. 424/141.1; 424/144.1

(57) ABSTRACT

A method of treating psoriasis and other inflammatory conditions of the skin by administering anti-CD40 molecules, such as mAb 5D12, in an amount sufficient to inhibit the immunological activation of keratinocytes. These anti-CD40 molecules include antibodies, peptides, and other molecules.

This application claims the benefit of priority of U.S. Provisional Application No. 60/198,174, filed Apr. 19, 2000, which is hereby incorporated by reference.

### FIELD OF THE INVENTION

The invention relates to CD40 antagonists for treating psoriasis and other inflammatory conditions of the skin.

### BACKGROUND OF THE INVENTION

There are numerous skin conditions characterized by an increased immune response and/or abnormal antigen presentation in the dermis and epidermis. The physiologic mechanisms involved in the evolution of such inflammatory processes are poorly understood. However, it has become apparent that skin cells are important in the generation of a cutaneous inflammatory response (Kupper, “Immune and Inflammatory Processes in Cutaneous Tissues”, J. Clin. Invest., 86, pp. 1783-89 (1990)).

Proliferative skin diseases are widespread throughout the world and afflict millions of humans and their domesticated animals. One example of a disease associated with keratinocyte hyperproliferation is psoriasis, a genetically determined disease the incidence of which is about 2% of the US population. Many pathologic features of psoriasis can be attributed to alterations in the growth and maturation of epidermal keratinocytes. Extensive scaling and a thickened epidermis are clinical hallmarks of this disease (G. D. Weinstein and J. L. McCullough, Cell Proliferation Kinetics, p. 327-342). The clinical manifestations are caused by hyperproliferation of epidermal cells. This hyperproliferation is also seen in non-psoriatic skin of psoriatic patients, indicating that the genetic defect is also present in apparently “normal” skin cells of psoriatic patients (Id.).

The normal adult epidermal population contains 1-2% Langerhans' cells and about 98% keratinocytes. Keratinocytes and other nonhematopoietically-derived cells resident in skin contribute to immune homeostasis and can produce various cytokines which influence migration of T cells and expression of adhesion molecules.

Ordinarily, the immune system protects the body against foreign antigens, e.g., parasitic infection, viral and bacterial infections, etc. It is well established, however, that a number of disease states and/or disorders are a result of either abnormal or undesirable activation of immune responses.

Immune responses involve the recruitment and activation of a number of immune system effector cells, i.e., B- and T-lymphocytes, macrophages, eosinophils, neutrophils, in a process coordinated through a series of complex cell-cell interactions. B-lymphocytes (“B-cells”) play an important role during an in vivo immune response to an antigen. A typical scenario by which an immune response is mounted against a foreign protein is as follows: An antigen will bind to the surface of a B-cell and trigger a chain of reactions, including increased expression of class 11 major histocompatability complex (MHC) molecules. Protein antigens are internalized and bind to these class 11 MHC molecules, to be presented on the cell surface. This in turn causes helper T-cell antigen recognition and activation. The activated T-cell expresses cell surface molecules, one of which is CD40 ligand (“CD40L”). CD40L binds to CD40, a 50 kDA type 1 membrane glycoprotein expressed on the surface of B-cells, causing the B-cell to mature and begin secreting soluble immunoglobulin.

It has recently been found that functional CD40 is expressed on a variety of cell types other than B-cells, including macrophages, dendritic cells, thymic epithelial cells, Langerhans cells, and endothelial cells. These studies have led to the current belief that CD40 plays a broad role in immune regulation by mediating interactions of T-cells with B-cells, as well as other cell types. In support of this notion, it has been shown that stimulation of CD40 in macrophages and dendritic results is required for T-cell activation during antigen presentation [Gruss et al., Leuk. Lymphoma, 24, 393 (1997)]. Recent evidence points to a role for CD40 in tissue inflammation as well. Production of the inflammatory mediators IL-12 and nitric oxide by macrophages have been shown to be CD40 dependent [Buhlmann and Noelle, J. Clin. Immunol., 16, 83 (1996)]. In endothelial cells, stimulation of CD40 by CD40L has been found to induce surface expression of E-selectin, ICAM-1, and VCAM-1, promoting adhesion of leukocytes to sites of inflammation [Buhlmann and Noelle, J. Clin. Immunol., 16, 83 (1996); Gruss et al., Leuk. Lymphoma, 24, 393 (1997)].

Studies have shown that B-cell functions, including proliferation, differentiation, rescue from apoptosis, and isotype switching, are induced when CD40 binds to CD40L. Cross-linking of CD40 molecules with anti-CD40 antibodies known in the art resulted in B cell activation. J. Banchereau et al., Science (1989) 147:8, demonstrated that anti-CD40 monoclonal antibodies (mAb) can mimic the effects of T helper cells in B-cell activation and induced B-cell proliferation. However, these antibodies were only stimulating B-cells and not inhibiting their proliferation or differentiation.

Recently, antibodies have been developed which bind to CD40 and do not stimulate the growth and differentiation of B cells, but instead inhibit B cell responses. See U.S. Pat. Nos. 5,677,165 and 5,874,082.

The in vivo role of the CD40/CD40L interaction has been demonstrated in animal models using anti-CD40L treatment, CD40 or CD40L knockout animals, or animals transgenic for CD40L expression. As expected, interference with this interaction reduces signs and symptoms of collagen arthritis, lupus, nephritis, graft-versus-host disease, experimental allergic encephalomyelitis (“EAE”) and allergic contact dermatitis, as well as increasing the survival of allografts.

Thus, interference with CD40 activity is potentially beneficial for antibody-mediated diseases such as autoimmunity, allergic diseases, and conditions in which immunogenic proteins are used therapeutically, such as in treatment with exogenous blood products or in gene therapy. Interference with CD40 activity could therefore be beneficial in treatment of cell-mediated immunological diseases, including psoriasis and other inflammatory conditions of the skin.

### SUMMARY OF THE INVENTION

The invention relates to agents and methods of inhibiting the activation of keratinocytes for the treatment of psoriasis or other inflammatory skin conditions by targeting, binding, or interacting with a particular epitope or epitopes on CD40, thereby inhibiting growth, activation, and/or differentiation of keratinocytes. The agents may have the additional property of not interfering with binding of CD40L to such epitope.

One example of such an epitope on CD40 is that bound by the antibody designated 5D12. This epitope is at amino acid residue numbers 52-63 of the CD40 antigen sequence (See SEQ ID NO:1). A model of the CD40 antigen shows that this epitope is on the opposite side of CD40 from where the CD40 ligand binds. Amino acids implicated in the binding of CD40L binding are located in the region of amino acid residue numbers 70 to 120 of CD40. See FIG. 1.

The molecules of the invention include monoclonal antibodies, fragments thereof, peptides, oligonucleotides, and other chemical entities. Also included are peptides and genes inducing expression of anti-CD40 antibodies. These molecules are useful for interrupting the CD40/CD40L interaction and in treatment of psoriasis and other inflammatory conditions of the skin.

### BRIEF DESCRIPTION OF THE DRAWINGS

FIG. 1 shows, in schematic form, the putative binding site of the monoclonal antibody 5D12, and the CD40L binding site on CD40.

FIG. 2 is a FACS graph showing that a saturating amount of antibody 5D12 does not affect binding of CD40L-FITC.

FIG. 3 is a FACS graph showing that pre-incubation of B cells with anti-CD40 antibodies other than 5D12 can prevent binding of CD40L-FITC.

### DETAILED DESCRIPTION OF THE INVENTION

The molecules described and used to inhibit activation of keratinocytes include monoclonal antibodies, fragments thereof, peptides, oligonucleotides and other chemical entities. Monoclonal antibodies can be made by the conventional method of immunization of a mammal, followed by isolation of the B cell producing the monoclonal antibodies of interest and fusion with a myeloma cell. The preferred monoclonal antibodies include chimeric antibodies, humanized antibodies, human antibodies, Delmmunised™ antibodies, single-chain antibodies and fragments, including Fab, F(ab′)₂, Fv and other fragments which retain the antigen binding function of the parent antibody. Single chain antibodies (“ScFv”) and the method of their construction are described in U.S. Pat. No. 4,946,778.

Chimeric antibodies are produced by recombinant processes well known in the art, and have an animal variable region and a human constant region. Humanized antibodies correspond more closely to the sequence of human antibodies than do chimeric antibodies. In a humanized antibody, only the complementarity determining regions (CDRs), which are responsible for antigen binding and specificity, are non-human derived and have an amino acid sequence corresponding to the non-human antibody, and substantially all of the remaining portions of the molecule (except, in some cases, small portions of the framework regions within the variable region) are Z human derived and have an amino acid sequence corresponding to a human antibody. See L. Riechmann et al., Nature (1988) 332: 323-327; U.S. Pat. No. 5,225,539; U.S. Pat. Nos. 5,585,089; 5,693,761; 5,693,762.

Human antibodies can be made by several different methods, including by use of human immunoglobulin expression libraries (Stratagene Corp., La Jolla, Califormia;

Cambridge Antibody Technology Ltd., London, England) to produce fragments of human antibodies (V_{H}, V_{L}, Fv, Fd, Fab, or (Fab′)₂), and use of these fragments to construct whole human antibodies by fusion of the appropriate portion thereto, using techniques similar to those for producing chimeric antibodies. Human antibodies can also be produced in transgenic mice with a human immunoglobulin genome. Such mice are available from Abgenix, Inc., Fremont, Califormia, and Medarex, Inc., Annandale, N.J. In addition to connecting the heavy and light chain Fv regions to form a single chain peptide, Fab can be constructed and expressed by similar means (M. J. Evans et al., J. Immunol. Meth. (1995) 184: 123-138).

Delmmunised™ antibodies are antibodies in which the potential T cell epitopes have been eliminated, as described in International Patent Application PCT/GB98/01473. Application of these antibodies in vivo is expected to eliminate or substantially reduce antibody immunogenicity in humans.

All of the wholly and partially human antibodies described above are less immunogenic than wholly murine or non-human-derived antibodies, as are the fragments and single chain antibodies. All these molecules (or derivatives thereof) are therefore less likely to evoke an immune or allergic response. Consequently, they are better suited for in vivo administration in humans than wholly non-human antibodies, especially when repeated or long-term administration is necessary, as may be needed for treatment of psoriasis or other inflammatory skin conditions.

Non-antibody molecules can be isolated or screened from compound libraries by conventional means. An automated system for generating and screening a compound library is described in U.S. Pat. Nos. 5,901,069 and 5,463,564. A more focused approach involves three-dimensional modeling of the binding site, and then making a family of molecules that fit the model. These are then screened for those with optimal binding characteristics.

Another approach is to generate recombinant peptide libraries, and then screen them for those that bind to the epitope of CD40 of interest. See, e.g., U.S. Pat. No. 5,723,322. Molecules can, in fact, be generated or isolated with relative ease in accordance with techniques well known in the art once the epitope is known.

Another approach is to induce endogenous production of the desired anti-CD40 antibodies, by administering a peptide or an antibody that induces such production, or through gene therapy, where a gene encoding anti-CD40 or a fragment thereof is administered, taken up intracellularly, and then expressed. The method of making and administering any of these molecules is well known in the art.

The molecules can be administered by any of a number of routes and are administered at a concentration that is therapeutically effective to prevent or treat psoriasis or other inflammatory skin conditions. To accomplish this goal, the antibodies may be formulated using a variety of acceptable excipients known in the art. Typically, the antibodies are administered by injection, either intravenously or intraperitoneally. Methods to accomplish this administration are known to those of ordinary skill in the art. It may also be possible to obtain compositions which may be topically or orally administered, or which may be capable of transmission across mucous membranes.

Before administration to patients, formulants may be added to the antibodies. A liquid formulation is preferred. For example, these formulants may include oils, polymers, vitamins, carbohydrates, amino acids, salts, buffers, albumin, surfactants, or bulking agents. Preferably carbohydrates include sugar or sugar alcohols such as mono, di, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. Sucrose is most preferred. “Sugar alcohol” is defined as a C₄ to C₈ hydrocarbon having an —OH group and includes galactitol, inositol, mannitol, xylitol, sorbitol, glycerol, and arabitol. Mannitol is most preferred. These sugars or sugar alcohols mentioned above may be used individually or in combination. Amino acids may include levorotary (L) forms of carnitine, arginine, and betaine; however, other amino acids may be added. Polymers may include polyvinylpyrrolidone (PVP) with an average molecular weight between 2,000 and 3,000, or polyethylene glycol (PEG) with an average molecular weight between 3,000 and 5,000. It is also preferred to use a buffer in the composition to minimize pH changes in the solution before lyophilization or after reconstitution. Most any physiological buffer may be used, but citrate, phosphate, succinate, and glutamate buffers or mixtures thereof are preferred. Most preferred is a citrate buffer. Preferably, the concentration is from 0.01 to 0.3 molar. Surfactants that can be added to the formulation are shown in EP Nos. 270,799 and 268,110.

Additionally, antibodies can be chemically modified by covalent conjugation to a polymer to increase their circulating half-life, for example. Preferred polymers, and methods to attach them to peptides, are shown in U.S. Pat. Nos. 4,766,106; 4,179,337; 4,495,285; and 4,609,546 which are all hereby incorporated by reference in their entireties. Preferred polymers are polyoxyethylated polyols and polyethylene glycol (PEG). PEG is soluble in water at room temperature and has a preferred average molecular weight between 1000 and 40,000, more preferably between 2000 and 20,000, most preferably between 3,000 and 12,000.

Water-soluble polyoxyethylated polyols may also be useful. They include polyoxyethylated sorbitol, polyoxyethylated glucose, and polyoxyethylated glycerol (POG). POG is preferred. One reason is because the glycerol backbone of polyoxyethylated glycerol is the same backbone occurring naturally in, for example, animals and humans in mono-, di-, triglycerides. Therefore, this branching would not necessarily be seen as a foreign agent in the body. The POG has a preferred molecular weight in the same range as PEG. The structure for POG is shown in Knauf et al., 1988, J. Bio. Chem. 263:15064-15070, and a discussion of POG/IL-2 conjugates is found in U.S. Pat. No. 4,766,106, both of which are hereby incorporated by reference in their entireties.

Additional pharmaceutical vehicles could be used to control the duration of action of the molecules of the invention. They could be entrapped in microcapsules prepared by coacervation techniques or by interfacial polymerization (hydroxymethylcellulose or gelatin microcapsules) in colloidal drug delivery systems (for example, liposomes, albumin microspheres, microemulsions, nano-particles and nanocapsules) or in macroemulsions. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example), which may include additional ingredients. Upon reconstitution, the composition is administered to subjects.

A preferred route of administration is parenterally. In parenteral administration, the compositions of this invention will be formulated in a unit dosage injectable form such as a solution, suspension or emulsion, in association with a pharmaceutically acceptable parenteral vehicle. Such vehicles are inherently nontoxic and nontherapeutic. Examples of such vehicles are saline, Ringer's solution, dextrose solution, and Hanks'solution. Nonaqueous vehicles such as fixed oils and ethyl oleate may also be used. A preferred vehicle is 5% dextrose in saline. The vehicle may contain minor amounts of additives such as substances that enhance isotonicity and chemical stability, including buffers and preservatives. Non-peptide molecules of the invention could be administered orally, including by suspension, tablets and the like. Liquid formulations could be administered by inhalation of lyophilized or aerosolized microcapsules. Suppositories could also be used.

The dosage and mode of administration will depend on the individual. Generally, the compositions are administered so that antibodies are given at a dose between 1 μg/kg and 20 mg/kg, more preferably between 20 μg/kg and 10 mg/kg, most preferably between 1 and 7 mg/kg. The dosage can be determined by routine experimentation in clinical trials, the starting point for which is a determination of optimal dosage by extrapolation from animal models in which the antibody was effective. is the antibody may be given as a bolus dose, to increase circulating levels by 10-20 fold and for 4-6 hours after the bolus dose. Continuous infusion may also be used following the bolus dose.

Such a dose ranging study could also monitor a variety of indicators related to the CD40-CD40L pathway, including a decrease in B-lymphocytes, monocytes or dendritic cells, or a decrease in free immunoglobulin and effect on disease symptoms. Adverse effects and side effects would also be monitored.

The in vivo effect of the molecules of the invention can be extrapolated from the known effects of certain anti-CD40 antibodies, which do not cause proliferation or differentiation of cells carrying CD40, including keratinocytes. The anti-CD40 monoclonal antibody designated 5D12 has been studied for effect on keratinocyte activation, as described below.

1. Location of the 5D12 Binding Epitope and the CD40L Binding Epitope

The reactivity of 5D12 with a panel of overlapping synthetic peptides corresponding to the amino acids sequence of the extracellular domain of CD40 was tested. Because Mab 5D12 binds poorly to CD40 when tested in Western blotting, some control experiments were performed to see if 5D12 would still bind to denatured CD40 in an ELISA system. CD40-Ig was coated onto ELISA plates by drying at 37° C. overnight or by incubation overnight in PBS at 4° C. In each case, CD40-Ig was pre-treated by boiling for 10 minutes and/or with 1 mM DTT.

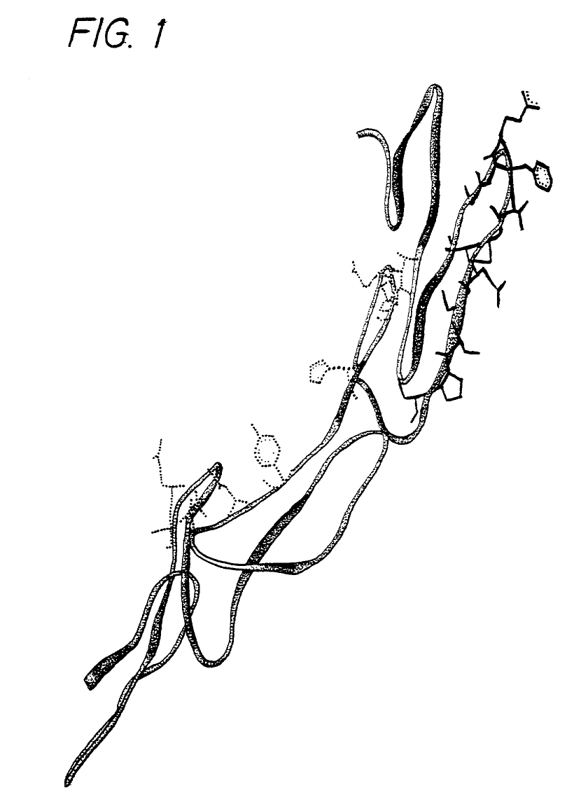

These pilot experiments demonstrated that boiling the antigen before coating did not significantly decrease the binding of Mab 5D12. However, reduction of all disulphide bonds in CD40-Ig strongly reduced the binding of Mab 5D12. Since a weak signal remained under these conditions, it was decided to proceed with the Pepscan analysis, which showed that Mab 5D12 strongly reacted with one specific 12-mer peptide of the extracellular part of CD40. (See SEQ ID NO:2) This peptide corresponds to amino acids 32 to 43 of the mature protein. At this position in the CD40 sequence there is a high degree (90%) of homology with CD40 from non-human primate species. In contrast, the degree of homology with mouse and bovine CD40, to which 5D12 does not bind, is only 42% and 58%, respectively. Interestingly, this peptide is distantly located from the CD40L binding site on CD40. Amino acids in CD40 that have been implicated in binding to CD40L are located in the region of amino acids 70 to 120 of the mature protein. It appears that the CD40L-CD40 interaction is concentrated on at least two clusters of residues on CD40 and it is predicted that CD40L-CD40 contacts are formed along the interface of two CD40L monomers with one CD40 chain (Bajorath et al., Biochemistry 34:9884 (1995)). FIG. 1 shows the location of the putative 5D12 binding epitope on a model of the extracellular domain of CD40 (Bajorath and Aruffo, Proteins 27:59 ((1997)). In this model the amino acids 32-43 are highlighted in bold and a number of residues postulated to be involved with CD40L binding indicated by dotted lines. This model clearly demonstrated that the putative CD40 binding epitope is located on the “outside” of the CD40 molecule, the “outside” being based on the hypothesis that three CD40 monomers bind around one CD40L trimer.

2. CD40L Binds to Another Location on CD40 from 5D12; 5D12 Seems to Affect CD40L Signaling

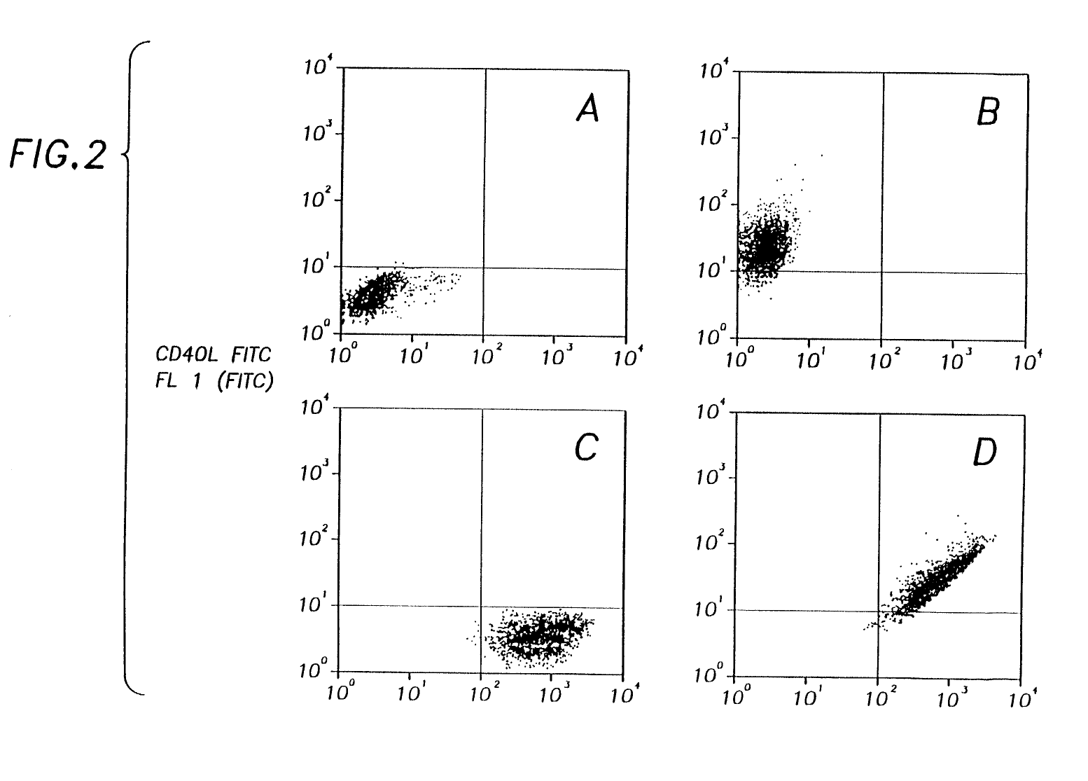

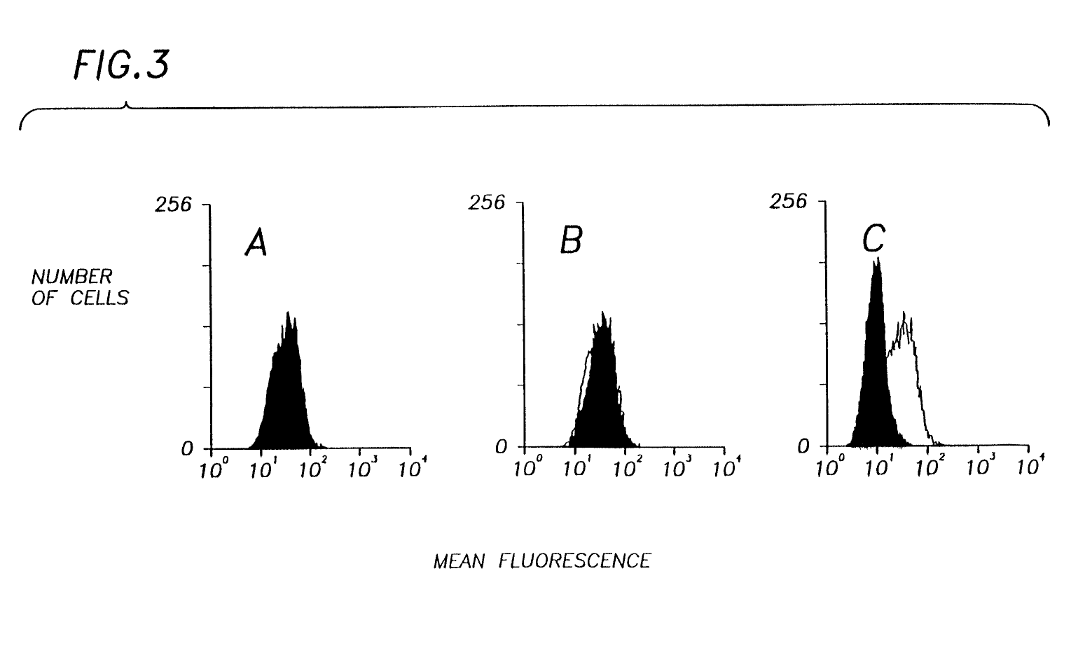

Preliminary two-color FACS analysis showed that 5D12 and a FITC-labeled soluble trimeric CD40L (CD40L-FITC) could simultaneously bind to CD40-expressing cells. Additional experiments were performed to test the hypothesis that 5D12 binds to a distinct epitope from that of CD40L. Pre-incubation of JY B cells with or without CD40L-FITC did not affect the staining intensity obtained with a saturating amount of 5D12. The reciprocal experiment showed that a saturating amount of 5D12 did not affect subsequent binding of CD40L-FITC (FIG. 2). In contrast, pre-incubation of JY B cells with other anti-CD40 monoclonal antibodies (one of which is designated G28.5) could prevent subsequent binding of CD40L-FITC (FIG. 3).

The disappearance of 5D12 and CD40L from stained JY B cells was investigated over time. When JY B cells labeled with CD40L-FITC were washed and subsequently cultured at 37° C., the fluorescent signal decreased over a period of hours. The release of CD40L-FITC from the cell surface was at about the same rate when the CD40L-FITC loaded cells were cultured in the presence of 5D12. Furthermore, in a reciprocal experiment, the level of CD40 on JY B cells did not appear to be significantly altered during culture with 5D12, nor did pre-binding CD40L-FITC to the cells affect the level of CD40 detected using 5D12.

In summary, these experiments clearly show that 5D12 in vitro: (i) does not compete with CD40L for binding to CD40; (ii) does not cause the release of CD40L bound to CD40; and (iii) does not cause modulation of CD40 from the cell surface. Previous results showed that the inhibitory effect of 5D12 on CD40 dominates over the stimulatory effect of CD40L. 5D12 may be modulating CD40 in such a way that signaling via CD40 is prevented or aborted when CD40L has already engaged CD40.

3. 5D12 Inhibits CD40L Mediated Activation

In a THP-1 assay, the effects of murine 5D12 on IL-8 production, induced by a number of different stimuli that are known to signal via NF□B, were tested. It was found that at concentrations where Mab 5D12 completely inhibits CD40L-mediated IL-8 production, there was no effect on IL-8 production by THP-1 cells stimulated with LPS, TNF-alpha, PMA or ionomycin, which normally induce IL-8 production.

4. Anti-CD40 Inhibits Activation of Keratinocytes

Keratinocytes are CD40 expressing immunocompetent cells. It is believed that in some inflammatory conditions of the skin keratinocytes express increased amounts of CD40 and may ligate with CD40L expressing activated T cells. This ligation may induce release of some inflammatory mediators and may thus participate in some inflammatory conditions of the skin. ELISA was used to test whether CD40 activation of IFN-γ pre-treated cultured human keratinocytes (CD40+ keratinocytes) by means of CD40L transfected cells or soluble CD40L can result in enhanced production of chemokines IL-8, RANTES and MCP-1 and of complement proteins C3 and factor B. Also tested was the effect of CD40 activation of CD40+ keratinocytes on the expression of the complement regulatory proteins: membrane cofactor protein (“MCP”), decay accelerating factor (“DAF”) and CD59 by flow cytometry.

CD40 activation of CD40+ keratinocytes up-regulated the release of IL-8 and RANTES greatly, and that of MCP-1 moderately. The production of C3 and factor B and the expression of MCP, DAF, and CD59 were not altered. Specificity of the results with CD40L transfected cells was confirmed using untransfected cells as controls, co-culturing CD40+ keratinocytes and transfected cells with and without physical contact with each other in a Transwell system, and inhibiting CD40 activation with neutralizing anti-CD40 monoclonal antibodies.

These experiments demonstrate that anti-CD40 molecules are effective in inhibiting activation of keratinocytes. Such molecules would be an effective treatment for psoriasis or other inflammatory skin conditions.

It should be understood that the terms and expressions used herein are exemplary only and not limiting, and that the scope of the invention is defined only in the claims which follow, and includes all equivalents of the subject matter of those claims.

We claim:

1. A method of inhibiting the immunological activation of keratinocytes comprising administering a molecule that binds to CD40 but does not activate CD40-expressing keratinocytes.

2. The method of claim 1, wherein the molecule does not alter expression of C3, factor B, MCP, DAF, or CD59.

3. The method of claim 1, wherein the molecule targets, binds to, or interacts with the epitope represented by SEQ ID NO:2.

4. The method of claim 1, wherein the molecule binds to CD40 but do not interfere with the binding of CD40L to CD40.

5. The method of any of claims 1 to 4, wherein the molecule is a monoclonal antibody or fragment thereof.

6. The method of claim 5, wherein the monoclonal antibody is chimeric, humanized, human, Delmmunised™ or single chain antibody.

7. A method of inhibiting the immunological activation of keratinocytes comprising administering a peptide, an antibody or a fragment thereof which induces endogenous production of anti-CD40 antibodies, or a gene coding for an anti-CD40 antibody or a fragment thereof.

8. A method of treating psoriasis or an inflammatory skin condition comprising administering a molecule that binds to or interacts with CD40 in an amount sufficient to inhibit the immunological activation of keratinocytes.

9. The method of claim 8, wherein the molecule binds to or interacts with the epitope represented by SEQ ID NO:2.

10. The method of claim 8, wherein the molecule binds to CD40 but do not interfere with the binding of CD40L to CD40.

11. The method of any of claims 8-10, wherein the molecule is a monoclonal antibody or fragment thereof.

12. The method of claim 11, wherein the monoclonal antibody is chimeric, humanized, human, Delmmunised™ or single chain antibody.
